(19) United States
(12) Patent Application Publication
(10) Pub. No.: US 2016/0271398 A1
(43) Pub. Date: Sep. 22, 2016

(54) Feedthrough of an Implantable Medical Electronic Device, Method for Producing Same, and Implantable Medical Electronic Device

(71) Applicant: BIOTRONIK SE & Co. KG, Berlin (DE)

(72) Inventors: Daniel Kronmueller, Nuernberg (DE); Michael Arnold, Erlangen (DE); Lilli Fries, Stein (DE)

(21) Appl. No.: 15/051,859

(22) Filed: Feb. 24, 2016

Related U.S. Application Data
(60) Provisional application No. 62/135,711, filed on Mar. 20, 2015.

(51) Int. Cl. A61N 1/375; H01B 17/26

(52) U.S. Cl. CPC A61N 1/3754; H01B 17/26

(57) ABSTRACT

A feedthrough of an implantable medical electronic device, including a ceramic or glass insulating body, a feedthrough flange surrounding the insulating body, and at least one connection element penetrating through the insulating body for external connection of an electric or electronic component of the device, wherein the feedthrough flange is joined from a number of pre-formed parts.

### CROSS-REFERENCE TO RELATED APPLICATIONS

This patent application claims the benefit of co-pending U.S. Provisional Patent Application No. 62/135,711, filed on Mar. 20, 2015, which is hereby incorporated by reference in its entirety.

### TECHNICAL FIELD

The present invention relates to a feedthrough of an implantable medical electronic device and also to a device of this type. This device typically comprises a device housing, in which electronic and electrical function units are housed, a device head having at least one electrode or a line connection, and a feedthrough arranged between device housing and device head for at least one electrical conductor element connecting the electrodes or the line connection to a function unit. A feedthrough of this type comprises an insulating body, particularly formed from ceramic or glass, a feedthrough flange surrounding the insulating body, and at least one connection element penetrating through the insulating body for the external connection of an electrical or electronic component of the device. The present invention also relates to a method for producing such a feedthrough.

### BACKGROUND

Implantable devices of the above-mentioned type have long been used on a mass scale, in particular, as cardiac pacemakers or implantable cardioverters (especially defibrillators). However, said device may also be a less complex device, such as, for example, an electrode line or sensor line or even a cochlear implant.

Most implantable medical electronic devices of practical significance are intended to deliver electrical pulses to excitable body tissue via suitably placed electrodes. In order to perform this function, electronic/electrical function units for generating the pulses and for suitably controlling the pulse generation are housed in the housing of the device, and electrodes or connections are provided directly on the device externally for at least one electrode line, in the distal end portion of which the electrodes are attached to the tissue for pulse transmission.

The electronic/electrical function units in the device interior are to be connected to the outer electrodes or electrode line connections in such a way that ensures utterly and permanently reliable function under the special conditions of the implanted state. Furthermore, the feedthrough of such a device has to ensure that said device is sealed permanently under these conditions.

In particular, feedthroughs of which the main and insulating body consists substantially of ceramic or glass are known, wherein multilayer or multi-part superstructures have also been developed with use of metals or metal oxides and are used. Such known feedthroughs largely satisfy the requirements placed thereon.

It is conventional practice to mill feedthrough flanges from solid material. The flanges are milled on a multi-axis CNC machine. Complicated geometries with undercuts can be produced. Various tools (e.g., drills, milling cutters, slot cutters, radiused cutters, etc.) are used serially during production.

The machining time grows with increasing complexity of the component. Material consumption and machine time for producing a flange are very high. Depending on machining, short or long chips are produced. Since chip formation influences the roughness of the workpiece, suitable measures (e.g., high-pressure coolants, special filters) have to be taken in order to remove the chips from the workpiece and prevent damage to the surface. Cavities in the body are not possible.

A feedthrough flange can alternatively also be produced by means of an MIM (metal injection molding) method. In this method, a mixture of metal powder and organic binder is injected into a mold, demolded and then sintered. In this method, the flange is produced as a whole in one step. The production of undercuts or cavities in the flange is not possible due to the process. The workpiece must have drafts, and traces from the ejector and closing edges of the die can often be seen on the component.

It is also known to form feedthrough flanges by stamping and deep drawing. Depending on the physical properties of the sheet metal used, considerable restoring forces sometimes occur during the necessary forming steps, such that a number of forming steps usually have to be provided at differently set temperatures. Here, the number of necessary recrystallizing intermediate anneals is lower. Uniform heating is mandatory; inter alia, substances containing molybdenum sulphide are recommended as lubricants. In order to obtain the optical quality of the sheet metal, insert films made of plastic must be used. The method has become established as an economical method for the production of simple geometries (for example, pacemaker housings) made of titanium.

Formed sheet metals have high accuracy, but only low component complexity. Since the starting product is always a sheet metal, complex structures (for example, grooves, undercuts) can only be produced with effort. Tightness can only be ensured with difficulty in the case of an integrated manufacturing process, since the semi-finished product has to be folded or severely formed a number of times, such that leakage paths are produced in the structure or remain open between the individual sheet planes.

The present invention is directed toward overcoming one or more of the above-mentioned problems.

### SUMMARY

Based on the above, an object of the present invention is to specify an improved feedthrough of the type specified in the introduction that can be produced in a relatively simple and, thus, economical, manner with high precision, in particular, with relatively high complexity of the mold. Furthermore, a corresponding production method will be specified and an implantable medical electronic device that can be produced relatively easily and economically will be provided.

At least this object is achieved in terms of the device aspects thereof by a feedthrough having the features of claim 1, and by an implantable medical electronic device having the features of claim 15. In terms of the method aspects thereof, an object is achieved by methods having the features of claims 8-10. Expedient developments are specified in the respective dependent claims.

In accordance with the present invention, the feedthrough flange in the proposed feedthrough is joined from a number of pre-formed parts, wherein, in particular, at least two of the pre-formed parts are joined by means of an integrally bonded connection, in particular, a hard-soldered connection, or a laser-welded connection, or at least one of the pre-formed parts is a pre-stamped and bent and/or folded and/or deep-drawn sheet metal part.

In an embodiment of the present invention the sheet metal part is produced, in particular, from a titanium sheet, or titanium alloy sheet. More specifically, the feedthrough flange can comprise a number of pre-formed sheet metal parts of different material quality, in particular, formed from a titanium or titanium alloy sheet on the one hand with grade 3-4 and, on the other hand, with grade 1-2.

In further embodiments, the feedthrough flange in accordance with a first aspect comprises a multilayer sheet metal part composite. Here, at least one pre-formed sheet metal part with resilient properties can be incorporated, in particular, in the multilayer sheet metal part composite and, in particular, is in direct contact with the insulating body.

In further embodiments of the present invention, the surface of the pre-formed sheet metal part, or of at least one pre-formed sheet metal part, is structured in the joint region thereof, and in particular, is embossed in a furrowed or wafer-like manner.

The proposed inventive method comprises a step of laser welding two pre-formed parts of this feedthrough flange in vacuum or under inert gas. In an alternative embodiment, or also combined with the above-mentioned step of laser welding, the inventive method comprises a step of hard-soldering two pre-formed parts of the feedthrough flange by means of a gold solder or gold alloy solder, in particular, in a joining step contiguous with the integration of the insulating body in the feedthrough flange.

In accordance with a relatively independent method aspect of the present invention, at least one pre-formed sheet metal part is produced as master sheet, and further sheet metal parts are each positioned in relation to the master sheet and in particular are joined thereto.

In an embodiment of the aforementioned, relatively independent method aspects, clamp connections of suitably pre-formed parts of the feedthrough flange to one another and/or to the insulating body and/or to the device housing for correct positioning thereof, are used before and/or during the assembly of the feedthrough and/or connection thereof to the device housing.

In a further embodiment of the proposed inventive method, a post-treatment step for reconstruction of the passivation layer of the pre-formed part of the feedthrough flange is performed after the joining step, in particular, as wet-chemical etching.

In applications of the present invention of practical significance, the proposed medical electronic device is formed as a cardiac pacemaker or implantable cardioverter or as a cochlear implant. However, the present invention is not limited to these device applications, but in principle can be used also in other devices that comprise a generic feedthrough.

The flange sub-assembly consists of various punched, sheet metal and bent parts. These are punched out separately in high quantity from suitable titanium sheet (for example, grade 1). Depending on the application and function element, it is possible to use titanium of another quality (for example, grade 3 or 4) to produce parts or sheets that require a strong spring effect.

In particular, one or more of the following advantages can be achieved with the present invention, at least in certain embodiments (as explained further above by way of example):

minimization of material use,

reduction of the machine time for production of the feedthrough,

overall reduction of the production costs, also due to the possibility of economical production of feedthrough parts as stamped parts in high quantity and option for integration of a forming step in the punching step,

possibility of adding mold details (for example, a welded-in flange or a splash guard for the step of welding of the feedthrough into the housing),

possibility of an integration of resilient elements or of stops, bearing surfaces or clamping surfaces for temporary or additional fixing of the insulation body or implant housing,

modularization of the feedthrough design, with the effect of using certain modules in high quantities and of being able to obtain said modules at accordingly low prices, and/or

option of a more autonomous feedthrough design more independent of suppliers, with subsequent publication of details and reduction of the risk of a loss of expertise.

Further embodiments, features, aspects, objects, advantages, and possible applications of the present invention could be learned from the following description, in combination with the Figures, and the appended claims.

### DESCRIPTION OF THE DRAWINGS

Advantages and expedient features of the present invention will also emerge from the description of exemplary embodiments with reference to the Figures, in which:

FIG. 1 shows a schematic, partly cut illustration of an implantable medical electronic device.

FIG. 2 shows a schematic illustration (sectional view) of a feedthrough flange of conventional design.

FIG. 3 shows a perspective illustration of a feedthrough in accordance with a further exemplary embodiment of the present invention.

FIGS. 4A-4F show schematic cross-sectional illustrations in order to explain variants of the present invention.

### DETAILED DESCRIPTION

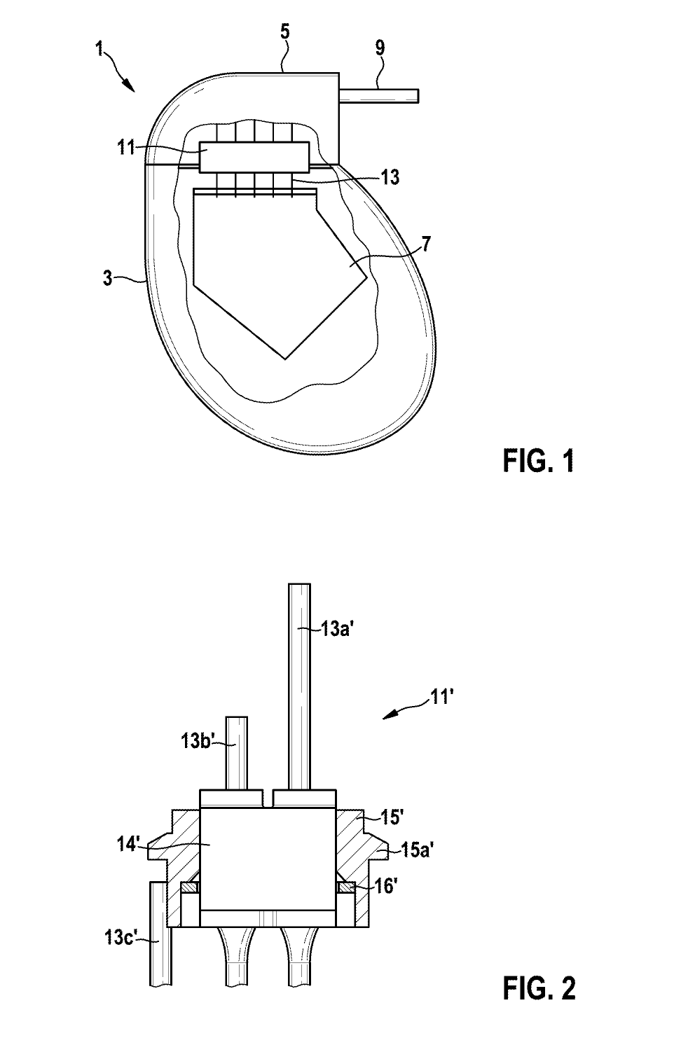

FIG. 1 shows a cardiac pacemaker 1 with a pacemaker housing 3 and a head part (header) 5, in the interior of which a printed circuit board (PCB) 7 is arranged, in addition to other electronic components, and an electrode line 9 being connected to the line connection (not shown) of said pacemaker, which line connection is arranged in the header. A feedthrough 11 provided between the device housing 3 and header 5 comprises a plurality of connection pins 13. The connection pins 13 are plugged at one end through a corresponding bore in the printed circuit board and are soft-soldered thereto.

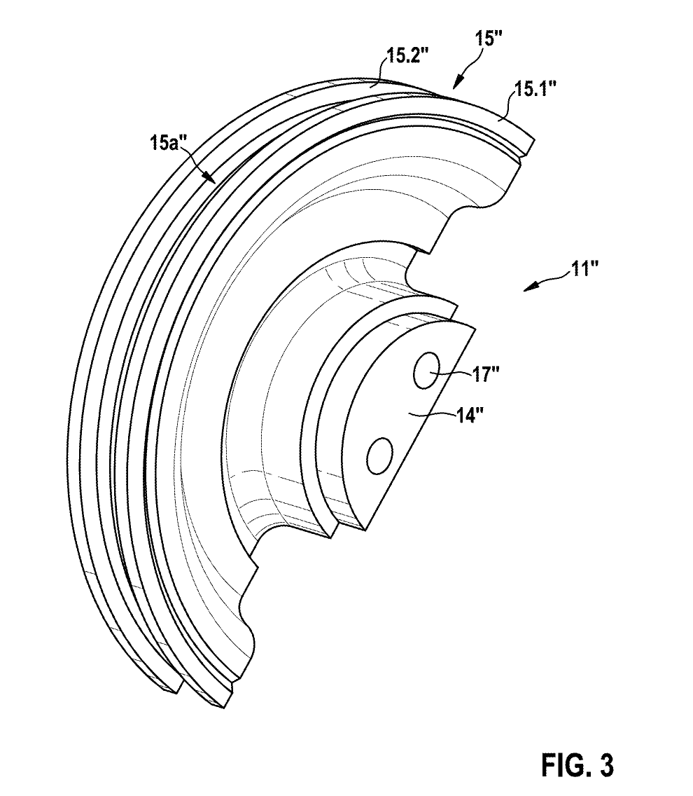

FIG. 2, in a sectional illustration along a central plane of section, shows a feedthrough 11′ with conventional structure, comprising a ceramic insulating body 14′ and a feedthrough flange 15′ formed by turning from solid material and surrounding the insulated body. A solder ring 16′ is inserted in a recess on the underside of the feedthrough flange 15′, said recess surrounding the insulating body annularly; there, the insulating body is connected in a hermetically sealed manner to this feedthrough flange by means of a hard soldering method. Long and short connection pins 13a′, 13b′ pass through the insulating body 14′, and a grounding pin 13c′ is welded externally onto the feedthrough flange 15′. A peripheral flange edge 15a′ on the feedthrough flange 15′ serves as a welding edge when the flange is inserted into a seat or bore of a device housing (not illustrated) and is welded there.

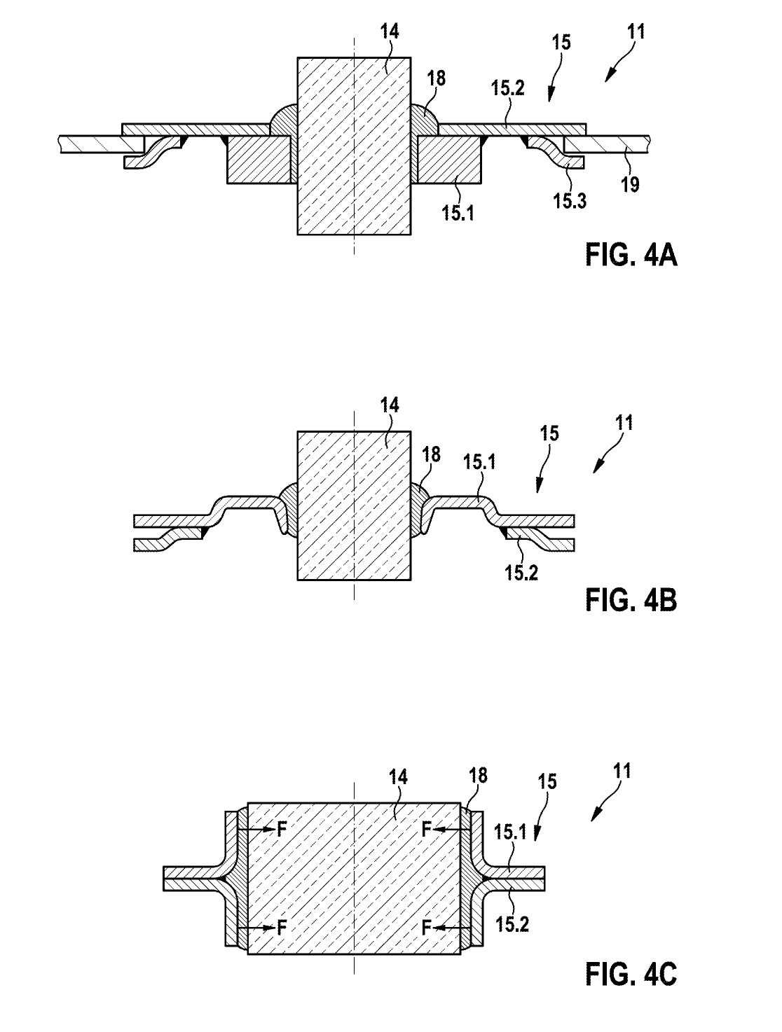

FIG. 3, as a perspective view, shows a feedthrough 11″ of a medical electronic device, said feedthrough being substantially plate-shaped in plan view and comprising a main and insulating body 14″ surrounded by a feedthrough flange 15″. Through-openings 17″ in the main and insulating body 14″ are provided in order to pass through connection pins (not shown). The feedthrough flange 15″ is assembled from an upper sheet metal part 15.1″ embossed in a complex basic mold and from a lower sheet metal part 15.2″, for example, by welding or hard soldering. The two sheet metal parts 15.1″, 15.2″ are shaped and joined together in such a way that they define a peripheral gap 15a″ there between at the outer periphery of the feedthrough 15″, which gap can be engaged by an inner peripheral edge of a device housing (not shown) of the medical electronic device, it also being possible for this housing to be welded here to the feedthrough.

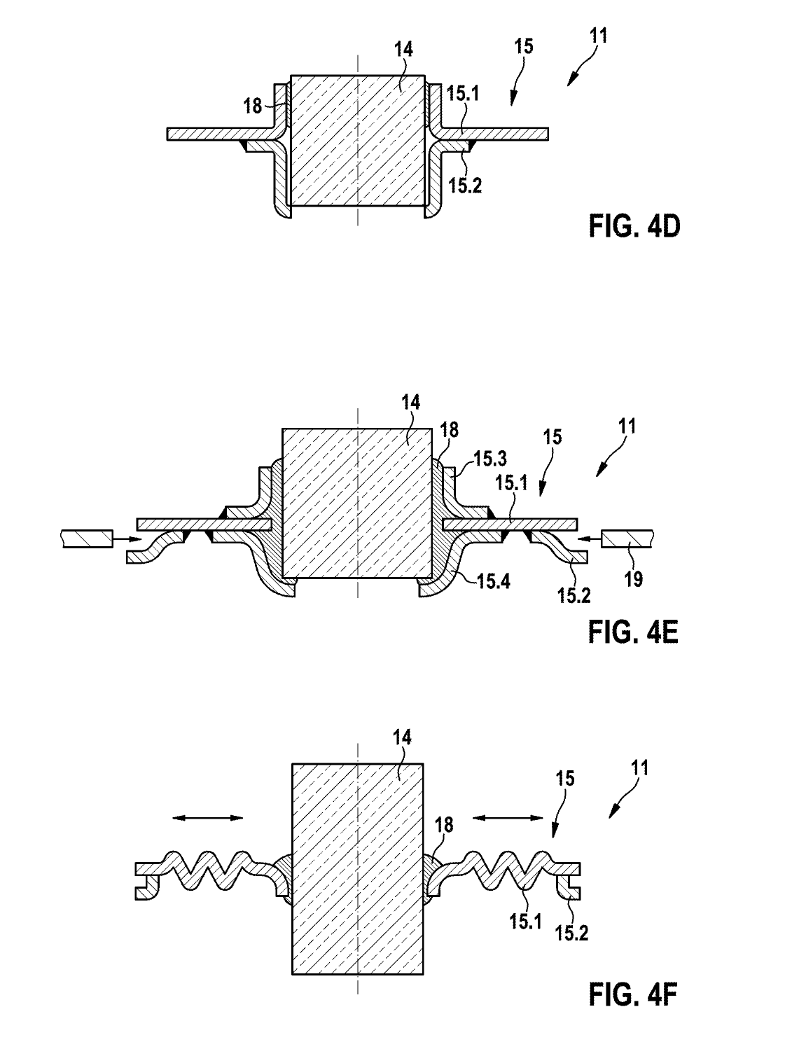

FIGS. 4A-4F show various embodiments or aspects of the present invention in a sketched manner in the form of longitudinal sectional illustrations of various feedthroughs. Although the feedthroughs sketched in these Figures in detailed views differ from one another, they are all denoted by reference numeral 11 as in FIG. 1, and the insulating bodies are denoted consistently by numeral 14 and the feedthrough flanges are denoted consistently by numeral 15. The insulating bodies are illustrated in each case in a simplified block-like manner; in practice one or more connection elements (connection pins) are usually embedded in said insulating bodies.

According to FIG. 4A, the feedthrough 11, besides the insulating body 14, also comprises a feedthrough flange 15 that is connected by means of a hard-soldered connection 18 to the insulating body and by means of a laser-welded connection (not shown) to a device housing 19. The feedthrough flange 15 is joined here from three parts, more specifically, a first flange part 15.1 closely surrounding the insulation body 15 annularly, a second flange part (sheet metal part) 15.2 welded or soldered thereto, and a third flange part (bent sheet metal part) 15.3 welded below the outer edge of said second flange part. The outer edges of the second and third flange part 15.2, 15.3 define a peripheral gap, with which the inner edge of the device housing 19 engages.

According to FIG. 4B, the feedthrough shown therein, besides the insulating body 14, also comprises a two-part feedthrough flange 15 that is joined from a first sheet metal part 15.1 and a second sheet metal part 15.2. The first sheet metal part 15.1 is bent a first time in the edge region thereof adjacent to the insulating body and a second time (in the opposite direction) at a distance therefrom, and the second sheet metal part 15.2 is joined to the first sheet metal part from below outside the second bend. Due to the first bend of the first sheet metal part 15.1, the contact surface with the soldered connection 18 is enlarged and, therefore, this connection can be produced more easily and with greater reliability.

FIG. 4C shows a further feedthrough 11, in which a comparable effect is attained in that here as well the feedthrough flange 15 is provided with an enlarged contact surface for the soldered connection 18. Here, this is achieved in that the flange is joined from a first and second sheet metal part 15.1, 15.2, which are folded in the inner edge region thereof in opposite directions. Due to this folding, a resilient contact pressure F of both sheet metal parts in the direction of the peripheral surface of the insulating body 14 is produced at the same time.

FIG. 4D shows a further embodiment of this design principle, wherein the second sheet metal part 15.2 is formed in such a way that the inner edge thereof surrounds the lower edge region of the insulating body 14 and thus produces an additional positioning and fixing effect.

FIG. 4E shows an embodiment that is similar to a certain extent to the embodiment according to FIG. 4, more specifically in particular in terms of the provision of a peripheral gap between a first, flat sheet metal part 15.1 and a second downwardly bent sheet metal part 15.2 joined to said first sheet metal part 15.1 in the outer edge region. In addition, in a development of the concept of the enlargement of the contact surface of the flange 15 with the soldered connection 18 sketched in FIGS. 4B-4D and described further above, a further sheet metal part 15.3, 15.4 is fitted on the inner edge of the first sheet metal part 15.1 below and above. Similarly to the embodiment according to FIG. 4D, the fourth sheet metal part 15.4 fitted below is formed such that it surrounds the lower peripheral edge of the insulating body 14 via a bent inner edge region.

FIG. 4F shows a feedthrough 11 of which the feedthrough flange 15 is joined from two sheet metal parts 15.1, 15.2, wherein the first part 15.1 is bent in a zigzagged manner and, thus, has resilience in the arrow direction, that is to say perpendicularly to the peripheral surface of the insulating body 14. With this shaping, the insulating body can be temporarily fixed in the feedthrough flange before the soldered connection 18 is produced.

Reference is made to the following embodiments of the present invention with regard to method aspects:

When producing the feedthrough flange from sheet metal parts these can initially be stamped in high quantity from sheet metal having suitable properties (for example, grade 1 titanium sheet). The parts are then formed subsequently or in the same production step. The necessary geometries (e.g., resilient elements, grooves, overlap joints) can thus be produced in a manner integrated into the sheets.

It is advantageous to produce a master sheet that is used to align and receive the other sheets. The greatest tolerances and critical functions here are ideally implemented already in the master sheet. The individual elements or sheets are then fitted in an automated manner onto the master sheet. Here, a suitable device or a manufacturing aid can be used for alignment. This ensures a uniform tolerance field or low form and position tolerances in relation to the maser sheet.

The sheets are joined (for example, spot welded) to one another or to the master sheet. In order to prevent an embrittlement or contamination of the material, titanium of the same type must be welded with exclusion of nitrogen, oxygen and hydrogen. It is therefore necessary for the sheets to be joined with inert gas (for example, argon min. 99.99%) or under vacuum. If function elements are fitted to the master sheet, a spot weld or butt joint is often sufficient. The solder for joining the ceramic insulator can be integrated by clamping between a number of sheets in the flange.

Auxiliary sheets with separating edges or predetermined break points can be joined on for the handling in the subsequent processes. The predetermined break points are dimensioned such that they are destroyed in the event of incorrect automated handling and, thus, prevent the automated assembly of damaged parts or the destruction of components during insertion.

Joint defects can be identified and rejected by an integrated optical inspection or a monitoring of the welding current.

During the joining, the natural passivation layer is destroyed. Scaling, annealing colors, deposits of metal oxides and slag can prevent a natural self-passivation, and may thus be seed points for subsequent corrosion. In order to provide the flange again with a protective passivation layer after joining, it is expedient to etch the component. Known etching solutions typically contain up to 4% hydrofluoric acid or 25% nitric acid.

The flange components can also be joined in part in the connection process during the high-temperature soldering. To this end, at least two flange components are produced and assembled. During the high-temperature soldering, they are joined by solder (for example, gold). As the flange elements are joined using gold they must be aligned with one another, positioned and fixed where appropriate. When joining the parts using hard solder, this is an integrated production step in the process chain. This means that there are no additional process steps. Since the simultaneous joining of a number of components in one process is complicated and the risk of production of excess is multiplied, this method is preferably suitable for components that are not critical (for example, fitting means, welded edges).

The present invention can also be realized in a large number of modifications of the examples shown here and aspects of the present invention underlined further above.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range. cm I/We claim:

1. A feedthrough of an implantable medical electronic device, comprising:
a ceramic or glass insulating body;
a feedthrough flange surrounding the insulating body; and
at least one connection element penetrating through the insulating body for external connection of an electric or electronic component of the device,
wherein the feedthrough flange is joined from a number of pre-formed parts.

2. The feedthrough according to claim 1, wherein at least two of the pre-formed parts are joined by means of an integrally bonded connection comprising a hard-soldered connection or a laser-welded connection.

3. The feedthrough according to claim 1, wherein at least one of the pre-formed parts is a pre-stamped and bent and/or folded and/or deep-drawn sheet metal part, and is formed from a titanium sheet or titanium alloy sheet.

4. The feedthrough according to claim 3, wherein the feedthrough flange comprises a number of pre-formed sheet metal parts of different material quality, and is formed from a titanium or titanium alloy sheet on the one hand with grade 3-4 and on the other hand with grade 1-2.

5. The feedthrough according to claim 3, wherein the feedthrough flange comprises a multilayer sheet metal composite.

6. The feedthrough according to claim 5, wherein at least one pre-formed sheet metal part with resilient properties can be incorporated in the multilayer sheet metal part composite and is in direct contact with the insulating body.

7. The feedthrough according to one of claim 3, wherein the surface of the pre-formed sheet metal part or of at least one pre-formed sheet metal part is structured in the joint region thereof, and is embossed in a furrowed or wafer-like manner.

8. A method for producing a feedthrough according to claim 1, comprising a step of laser welding two pre-formed parts of the feedthrough flange in vacuum or under inert gas.

9. A method for producing a feedthrough according to claim 1, comprising a step of hard-soldering two pre-formed parts of the feedthrough flange by means of a gold solder or gold alloy solder, in a joining step contiguous with the integration of the insulating body in the feedthrough flange.

10. A method for producing a feedthrough according to claim 1, wherein at least one pre-formed sheet metal part is produced as master sheet, and further sheet metal parts are each positioned in relation to the master sheet and are joined thereto.

11. The method according to claim 8, wherein clamp connections of suitably pre-formed parts of the feedthrough flange to one another and/or to the insulating body and/or to the device housing for correct positioning thereof are used before and/or during the assembly of the feedthrough and/or connection thereof to the device housing.

12. The method according to claim 9, wherein a post-treatment step for reconstruction of the passivation layer of the pre-formed part of the feedthrough flange is performed after the joining step as wet-chemical etching.

13. An implantable medical electronic device comprising a feedthrough according to claim 1.

14. The device according to claim 13, said device being formed as a cardiac pacemaker or implantable cardioverter.

15. The device according to claim 13, said device being formed as a cochlear implant.
